(19) United States
(12) Patent Application Publication
(10) Pub. No.: US 2026/0069221 A1
(43) Pub. Date: Mar. 12, 2026

(54) SMART SURVEILLANCE DEVICE

(71) Applicant: Emaww, Saint-Lambert (CA)

(72) Inventors: Alicia Heraz, Saint-Lambert (CA); Ashraf Nijim, Saint-Lambert (CA); Khaled Nigim, Saint-Lambert (CA)

(21) Appl. No.: 19/323,716

(22) Filed: Sep. 9, 2025

Related U.S. Application Data
(60) Provisional application No. 63/693,109, filed on Sep. 10, 2024.

(51) Int. Cl. A61B 5/00; A61B 5/01; A61B 5/11; A61B 5/16; G16H 50/30

(52) U.S. Cl. CPC A61B 5/746; A61B 5/01; A61B 5/1114; A61B 5/165; A61B 5/7203; A61B 5/7264; A61B 5/7405; A61B 5/747; G16H 50/30

(57) ABSTRACT

Methods, systems, and apparatus, including computer programs encoded on computer storage media, for monitoring physiological data. In some implementations, a system obtains, from a device, sensor data that includes physiological data of a user monitored by device. The system generates feature data from the obtained sensor data, the feature data configured to be processed by one or more trained machine learning models. The system provides the generated feature data as input to the machine learning models. The system obtains, from the trained machine learning models, output that represents the one or more health or behavioral metrics for the user. The system determines whether the obtained output that represents the one or more health or behavioral metrics for the user satisfies a corresponding threshold value. In response, the system performs an action for the user to mitigate the output that represents the one or more health or behavioral metrics.

### CROSS-REFERENCE TO RELATED APPLICATION

This application claims the benefit of U.S. Provisional Application No. 63/693,109, filed on Sep. 10, 2024, which is incorporated herein by reference in its entirety.

### TECHNICAL FIELD

This specification describes technologies to detect and monitor physiological conditions of users using an enclosed device.

### BACKGROUND

Physiological monitoring typically relies on the use of measuring, recording, and analyzing an individual's vital signs. This can include measuring the individual's health, detecting changes related to their health signs, and providing care related to their detected vital signs. However, monitoring an individual's vital signs can be limited in certain environments when the necessary equipment is unavailable to ensure such monitoring.

### SUMMARY

This specification describes techniques related to a monitoring system designed to enhance safety and health surveillance across sensitive environments. Specifically, the monitoring system includes an electronic smart device capable of monitoring individuals' physiological characteristics. These applicable environments may include, for example, secure facilities, detention cells, hospitals, mobile units, and other environments that require continuous observation. The smart electronic device can be placed strategically, such as out of reach of the individuals being monitored, to ensure tamper resistance while transmitting data for real time or substantial real time monitoring. The architecture enables reliable detection and differentiation of certain individual actions, such as, for example, destructive behaviors, self-harm, medical distress, and emotional states, while ensuring timely interventions, safeguarding privacy, and minimizing disruption to existing systems.

In some implementations, the smart electronic device can integrate multiple sensors within a compact housing for monitoring individuals. In some examples, the smart device can be constructed in the form of a connected pentahedron prism, a triangular prism, or another suitable geometric configuration. The smart device can be equipped with the multiple sensors to monitor a range of physiological characteristics that include, for example, a body position, body motion, a breathing rate, a heart rate, a respiration rate, and a current body temperature.

To ensure device durability and preserves an integrity of the smart electronic device, the smart electronic device may be mounted in certain locations within the environment. These locations can include, for example, wall-ceiling corners, ceilings, walls, or other locations. The smart electronic device can conceal not only the multiple sensors but also the wiring, which preserves the unobtrusiveness of the device in the environment. Once the smart electronic device is installed, the smart electronic device can continuously captures and process physiological data to detect self-harm, destructive behaviors, and health issues, of multiple users. The collected data is communicated to a remote station, which provides for a rapid safety and emergency response while preserving the anonymity of monitored individuals. This privacy approach ensures complains and ethical standards while safely monitoring individuals physiological conditions.

In one general aspect, a method is performed by one or more computers, such as a server. The method includes: obtaining, from a device, sensor data that comprises physiological data of one or more users monitored by the device; generating feature data from the obtained sensor data, the feature data comprising information corresponding to the physiological data and configured to be processed by one or more trained machine learning models; providing the generated feature data as input to the one or more trained machine learning models, wherein the one or more trained machine learning models are configured to process the information corresponding to the physiological data to generate one or more health or behavior metrics; obtaining, from the one or more trained machine learning models, output comprising information corresponding to the one or more health or behavioral metrics for the one or more users monitored by the device; determining whether the information corresponding to the one or more health or behavioral metrics for the one or more users satisfies a respective threshold value for each of the one or more users; in response to determining that at least a portion of the information does not satisfy the respective threshold value for at least one user of the one or more users, performing one or more actions for the at least one user, wherein the one or more actions are configured to address the one or more health or behavioral metrics for the at least one user; and generating an alert related to at least the portion of the information that does not satisfy the respective threshold for at least the one user of the one or more users.

Other embodiments of this and other aspects of the disclosure include corresponding systems, apparatus, and computer programs, configured to perform the actions of the methods, encoded on computer storage devices. A system of one or more computers can be so configured by virtue of software, firmware, hardware, or a combination of them installed on the system that in operation cause the system to perform the actions. One or more computer programs can be so configured by virtue having instructions that, when executed by data processing apparatus, cause the apparatus to perform the actions.

The foregoing and other embodiments can each optionally include one or more of the following features, alone or in combination. For example, one embodiment includes all the following features in combination.

In some implementations, obtaining sensor data that includes physiological data of one or more users monitored by the device includes obtaining, from the device, the sensor data that includes the physiological data of the one or more users monitored by the device, wherein the device comprises a multi-sensor device that is configured to (i) detect concealed objects on the one or more users, (ii) track body posture of the one or more users, and (iii) tracking the physiological data of the one or more users.

In some implementations, the physiological data of the one or more users includes a breathing rate, a heart rate, a respiration rate, and a body temperature.

In some implementations, generating feature data from the obtained sensor data includes applying one or more noise reduction algorithms to the obtained sensor data to reduce noise in the obtained sensor data that was created from an area outside where the one or more users are being monitored by the device.

In some implementations, providing the generated feature data as input to the one or more trained machine learning models includes: providing the generated feature data as input to at least one of a convolutional neural network (CNN), a reinforcement learning (RL) algorithm, or a long-short term memory (LSTM) model, wherein the CNN is configured to detect one or more actions indicative of a medical emergency associated with the one or more users using the generated feature data, wherein the RL algorithm is configured to generate feedback to improve conditions of one or more sensors of the device according to detected environmental conditions in the generated feature data, and wherein the LSTM model is configured to detect temperature anomalies in the generated feature data.

In some implementations, obtaining, from the one or more trained machine learning models, output including information corresponding to the one or more health or behavioral metrics for the one or more users monitored by the device includes obtaining data indicative of one or more of destructive behaviors, self-harm behaviors, or medical distress for each user of the one or more users.

In some implementations, determining whether the information corresponding to the one or more health or behavioral metrics for the one or more users satisfies a respective threshold for each of the one or more users includes: obtaining a likelihood for each of the one or more health or behavioral metrics for each of the one or more users; retrieving one or more thresholds for each of the one or more users; and comparing the likelihood for each of the one or more health or behavioral metrics for each of the one or more users to the respective threshold from the one or more thresholds.

In some implementations, the method further includes: determining that the likelihood of at least one of the one or more health or behavioral metrics for a user does not satisfy the respective threshold for the user; or determining that the likelihood for each of the one or more health or behavioral metrics does satisfy the respective threshold from the one or more thresholds.

In some implementations, performing one or more actions for the at least one user includes: opening a door at a location where the at least one user is being monitored; repeatedly turning a light off and on at the location where the at least one user is being monitored; transmitting a notification to the authorities to alert of an issue associated with the at least one user; or providing a message to the device that is monitoring the at least one user to cause the device to output an audible message through a speaker of the device.

In some implementations, the alert includes data identifying the one or more users, the sensor data of the one or more users, or the obtained information corresponding to the one or more health or behavior metrics for the one or more users monitored by the corresponding device.

In some implementations, generating the alert includes displaying the alert related to tracking the one or more users monitored by the device.

In some implementations, a device includes: one or more thermal cameras configured to generate thermal data of one or more users in a location monitored by the device; one or more ultrasonic sensors configured to analyze sound reflections to detect movement of the one or more users in the location; one or more infrared sensors configured to monitor physiological data of the one or more users; one or more microwave sensors configured to generate motion data and the physiological data of the one or more users; and a control unit configured to: generate combined data comprising the thermal data, the detected movement, the physiological data, and the motion data; and generate one or more messages characterizing the combined data.

In some implementations, the device further includes one or more acoustic sensors configured to record noise and sounds at the location of the one or more users.

In some implementations, the device further includes a housing configured to house the one or more thermal cameras, the one or more ultrasonic sensors, the one or more infrared sensors, the one or more microwave sensors, and the control unit.

In some implementations, a front panel of the housing includes at least one of: a transparent polycarbonate material configured to allow infrared transmissions, reinforced fiberglass, or reinforced polycarbonate.

In some implementations, the transparent polycarbonate material is a one-way transparent material.

In some implementations, the housing includes at least one of Aluminum Composite Panels (ACM) or Polycarbonate.

In some implementations, the housing includes a right triangular pentahedron prism.

In some implementations, the right triangular pentahedron prism is configured to mount to a wall-ceiling junction.

The subject matter described in this specification can be implemented in various embodiments and may result in one or more of the following advantages. In some implementations, the smart electronic device provides multi individual physiological monitoring through a fully housed sensor suite. The portable design of the smart electronic device facilitates the relocation between environments, such as detention cells, hospitals, children's daycare, without loss of functionality. This versatility ensures wide applicability where constant monitoring of individuals is critical.

In some implementations, the configuration of the sensors within the smart device enables the sensors to work collectively and to avoid interference among the sensors. In particular, certain sensors require unobstructed fields of view or non-overlapping power spectra. For example, the thermal cameras can be positioned at opposing ends of the housing to maximize environmental visibility, while the infrared, acoustic, microwave sensors are strategically positioned in between the thermal sensors to preserve their functionality. This coordinated configuration enables each of the sensors to function collectively interference.

Typically, operational policies do not permit guards within the holding cell area unless a police officer is present. However, in most remote and rural areas, an officer may not be present at the detachment. The smart device can provide early detection of concerning behaviors and will assist the guards in the performance of their duties. In particular, the smart device can provide time sensitive situations alerting if the person in-custody is experiencing life-threatening behavior or symptoms. The modernization of the approach to detainee monitoring with the implementation of advanced technology can reduce in cell death. Use of this smart device technology may also enable improved public trust of safeguarding individuals held in custody.

The smart device can detect, distinguish, and send an audible and visual alert when destructive behaviors are exhibited in the holding cell. This can include, for example, lighting fire, fighting, hitting cell fixtures, such as the cell door, or reaching their arm from under cell door into the corridor—to trip or grab a personnel). The smart device can detect, distinguish, and send an audible and visual alert when self-harm behaviors are exhibited in the holding cell. This can include, for example, attempting to strangle/hang oneself, attempting to drown oneself in the toilet, repetitive banging of ones' head on the cell wall and door, attempting to chip and eat paint or caulking, or attempt to cut oneself. The smart device can detect, distinguish, and send an audible and visual alert when medical distress is exhibited in the holding cell, e.g., monitoring and alerting life-threatening heart rate and breath rate above or below threshold, monitoring and alerting life-threatening respiratory rate, measuring and alerting for extreme body temperature (fever), monitoring perspiration (indication of diabetes, heart failure, anxiety, and overactive thyroid).

The smart device can be installed within the cell at a height of 2.9 m above the cell floor (or where wall meets ceiling) and have no exposed conduit. The smart device can be easily accessible for personnel to perform regular maintenance (e.g. cleaning) within a short period of time (e.g., less than one hour), as these cells are intended to remain operational continuously (e.g., 24 hours per day, 7 days a week). The smart device can be tested and approved by to ensure it is resistant to tampering (such as picking, tearing, or scratching), resistant to heat (i.e. from a lighter), resistant to 130 joules of impact from kicking or punching, not have any ligature (hanging) points, or the ability to create or conceal weapons and/or contraband.

The smart device can be installed with an approved protective cover that meets the requirements and have the ability to allow medical personnel to set heart rate and breath rate thresholds to meet the needs of individuals in-custody based on known factors or pre-existing conditions, such as diabetes, heart condition, etc. In some cases, the system can locally record and retain sensor data for post incident analysis and data retention in accordance with RCMP Information Management policies. The system can be contact-less and a non-wearable sensor (securely positioned in/near the cell and outside reach of the detainee) and include a user-friendly interface that is intuitive, easy to navigate, and efficient for personnel with limited computer experience.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram that illustrates an example of a system for monitoring individuals physiological conditions within an environment.

FIG. 2 illustrates an example of a smart cell equipped with a penta-box.

FIG. 3 illustrates an example of a penta-box contractible and extensible length.

FIG. 4 illustrates an example of the sensors included within the penta-box.

FIG. 5 illustrates an example configuration of the penta-box power and data cables connections.

FIG. 6 illustrates an example overall data flow and processing systems.

FIG. 7 is a flow diagram that illustrates a process for detecting an individual's physiological condition within an environment using the smart electronic device.

Like reference numbers and designations in the various drawings indicate like elements. The components shown here, their connections and relationships, and their functions, are meant to be examples only, and are not meant to limit the implementations described and/or claimed in this document.

### DETAILED DESCRIPTION

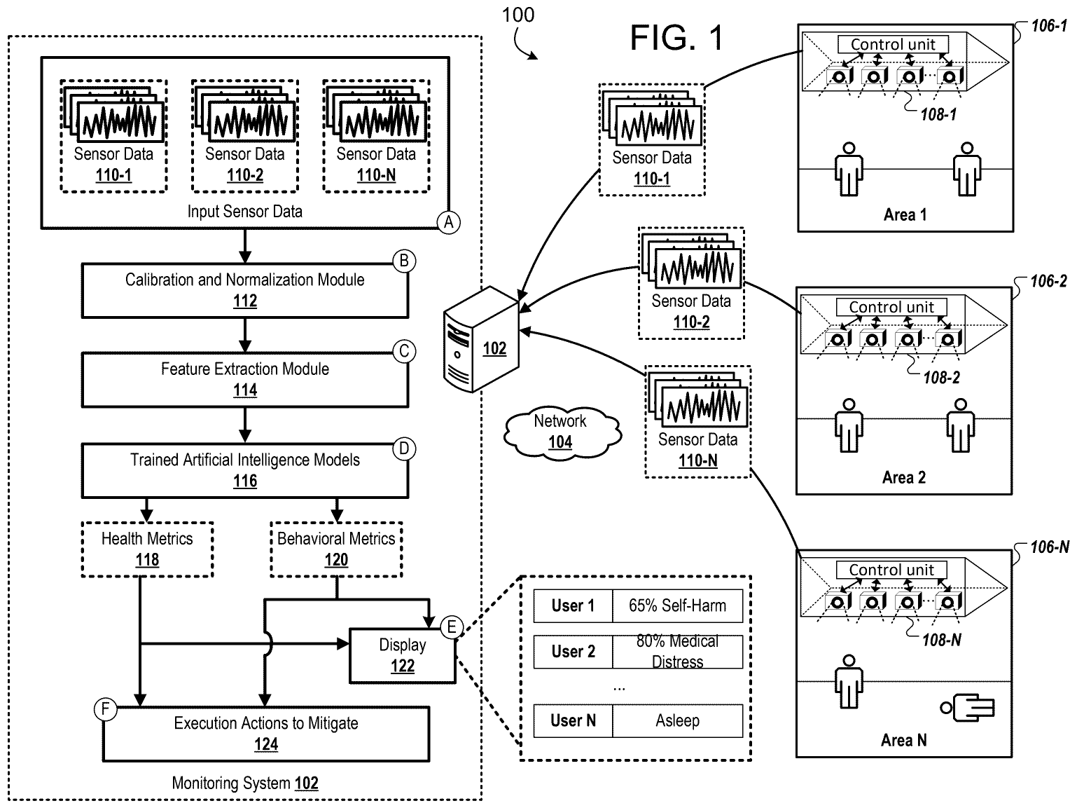

FIG. 1 is a block diagram that illustrates an example of a system 100 for monitoring individuals physiological conditions within an environment. The system 100 includes a monitoring system 102 and multiple environments 106-1 through 106-N (collectively “environments 106”). The system 100 can communicate with different electronic smart devices 108-1 through 108-N (collectively “smart devices 108”) utilized at each of the environments 106-1 through 106-N (collectively “environments 106”) over a network. The network 104 can include a wired network, a wireless network, a local network, or an external network, such as the Internet.

Briefly, the monitoring system 102 can receive sensor data from each of the smart devices 108 to predict physiological metrics of the users monitored by the smart devices 108. In some cases, the monitoring system 102 can calibrate and filter the received sensor data and provide the corresponding data as input to a trained machine learning model. The trained machine learning model can process the sensor data to produce detection of complex behavior and health indicators for each of the users, as will be further described below. For example, the trained machine learning model can include a convolutional neural network, which can be utilized to produce a detection of activity recognition, e.g., movement, and medical distress detection.

In some implementations, the monitoring system 102 can produce perform one or more additional actions based on the behavioral and health metrics. In some cases, the monitoring system 102 can provide one or more likelihoods indicative of the behavioral and health metrics to a display screen for real time or substantial real time review by a third party. In some cases, the monitoring system 102 can compare the one or more likelihood indicative of the behavioral and health metrics to user defined thresholds. If the monitoring system 102 determines the likelihoods satisfies the user defined thresholds, the monitoring system 102 determines the user is experiencing a behavioral or medical issue. In this case, the monitoring system 102 can execute one or more additional actions in attempt to mitigate the behavioral or medical issue. The additional actions may include, for example, opening a door to enable a user access to the user experiencing the behavioral or medical issue, flickering a light in the location where the user is located to attempt to wake the user, or alerting medical authorities of the behavioral or medical issue detected.

In some implementations, the monitoring system 102 can include one or more servers or computers connected locally or over a network. The monitoring system 102 can include a network 104 that can be, for example, a local network, a Wi-Fi network, an intranet, an Internet connection, a Bluetooth connection, or some other connection that enables the monitoring system 102 to communicate, e.g., transmit and receive, sensor data and other data with various databases and various computers or client devices.

In some implementations, each smart device 108 includes one or more sensors that monitors individuals. In particular, each smart device 108 is designed to improve safety and health surveillance within a particular environment, e.g., detention cells, hospitals, and other. The smart device 108 can be configured as a Penta Box, shaped in the form of a three dimensional pentagon. In some examples, the Penta-Box can include a durable right triangular pentahedron prism that houses the multiple sensors. In some examples, the Penta Box can include a durable housing that is constructed in another geometric configuration. The smart device 108 avoids visual cameras that can identify individuals, instead using non-intrusive sensors to detect and respond to behaviors. The use of microwave sensors for precise movement detection and AI models for pattern recognition ensures compliance with privacy standards.

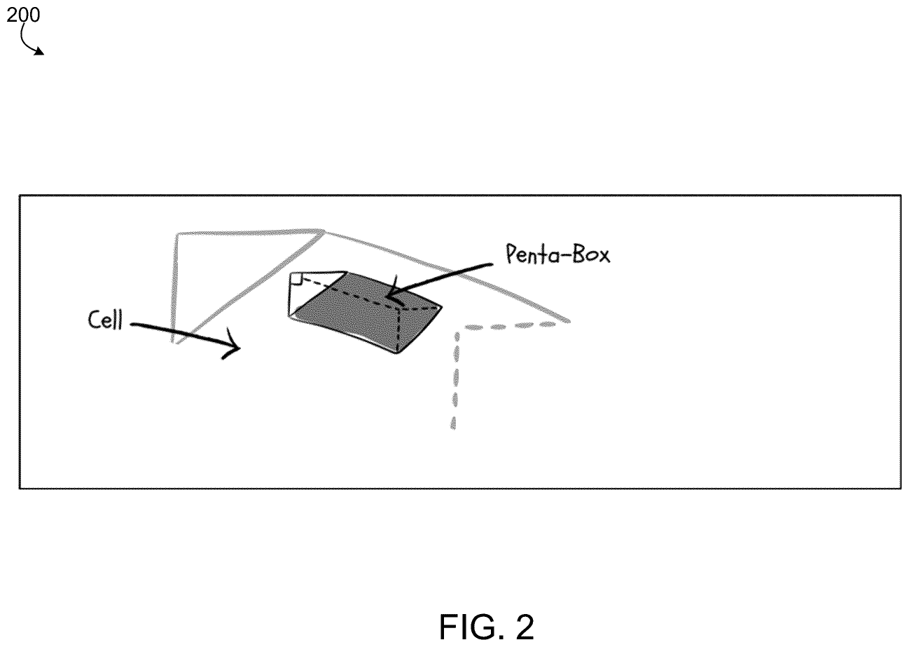

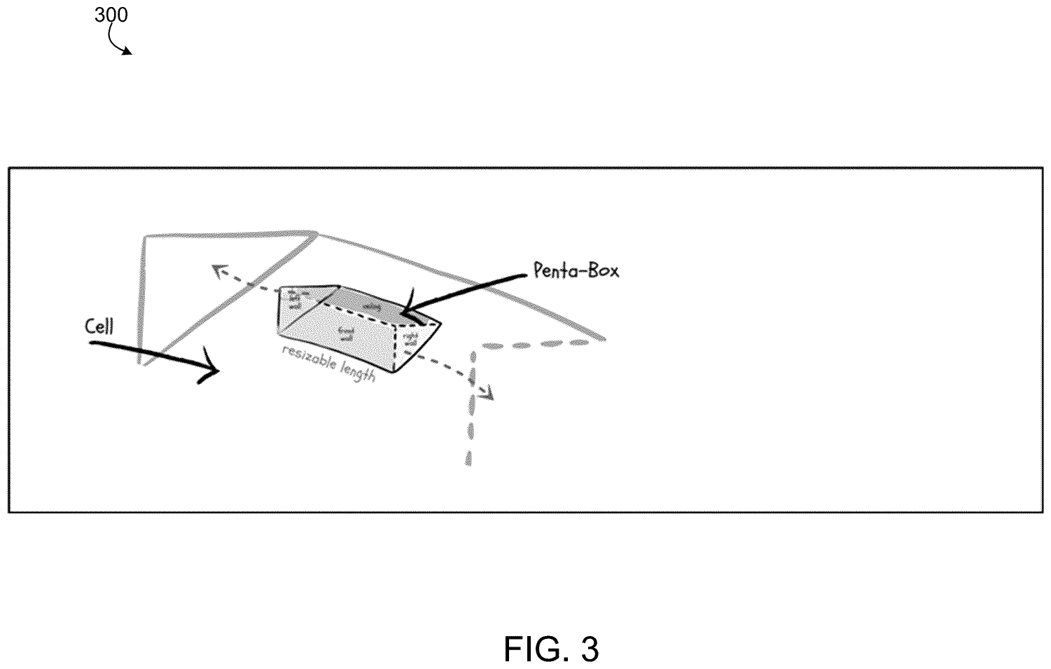

In some examples, as shown in FIG. 2, the Penta Box or smart device 108 can be enclosed within a smart cell housing. As shown in FIG. 3, the smart device 108 can be resizable to fit within the smart cell housing. In some examples, the smart cell housing may be a protectable housing of the smart device 108. In some examples, the sensors within the smart device 108 are concealed behind a one-way transparent indestructible cover.

As illustrated in system 100, the smart device 108 can be mounted to a location in the environment. For example, the smart device 108 can be mounted at a wall-ceiling junction, a ceiling, a wall, on a table, or located in another area. Generally, the smart device 108 is placed in an area that enables monitoring of individuals. In some cases, the smart device 108 can be placed in an area that prevents the individuals being monitored from accessing and obstructing the smart device 108 itself. For example, the smart device 108 can be secured to the wall using screws, fasteners, bolts, or any other attachments that allow secure coupling of the smart device 108 to a wall.

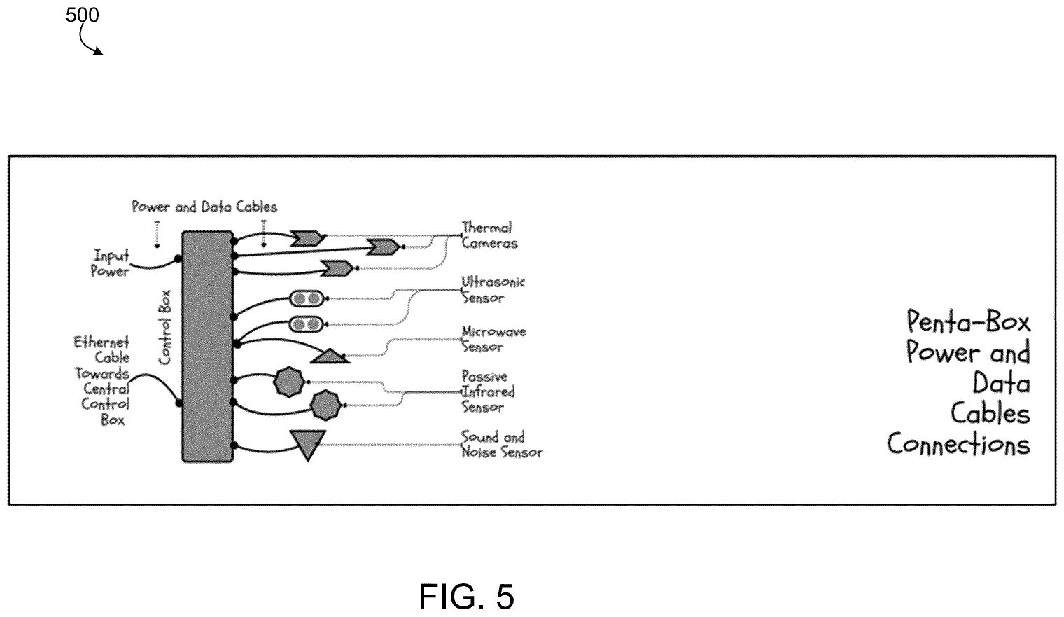

In some examples, the smart device 108 can be installed at a height of 2.9 meters, for example, with concealed wiring. The modular design facilitates easy maintenance and compliance with operational requirements. The control unit and sensors within the smart device 108 are connected to an emergency battery and input power that ensures continuous monitoring and data flow with no interruptions, as illustrated in FIG. 5.

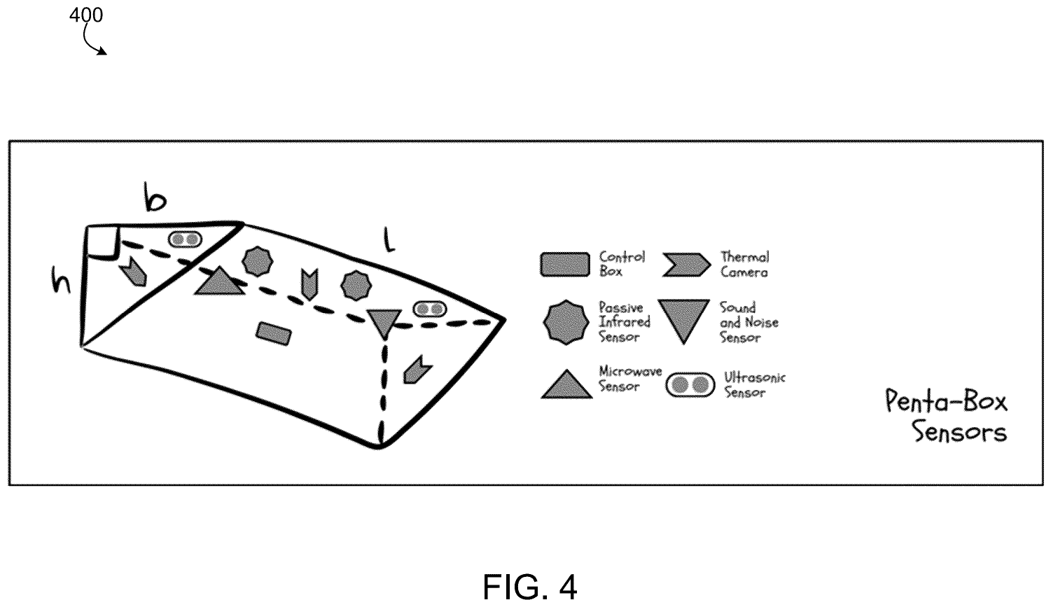

In some implementations, each smart device 108 can include multiple sensors. Each sensor of the multiple sensors is placed in the smart device 108 in a configuration that allows for each sensor to work collectively and avoid interference among the sensors. For example, each smart device 108 can include three thermal cameras, two ultrasonic sensors, two passive infrared sensors, one sound and noise sensor, one microwave sensor, and a control unit. The number of sensors may vary according to design specification and configuration. The thermal cameras, ultrasonic sensors, passive infrared sensors, acoustic sensors, e.g., sound and noise sensors, and microwave sensor provide their information to a control unit. One such configuration of the sensors within the smart device 108, as shown in FIG. 4, illustrates the thermal cameras on opposite of the smart device 108 and the remaining sensors in between. The control unit of each smart device 108 can communicate with the central server or monitoring system 102 over network 104 using a transceiver, as illustrated in FIG. 1.

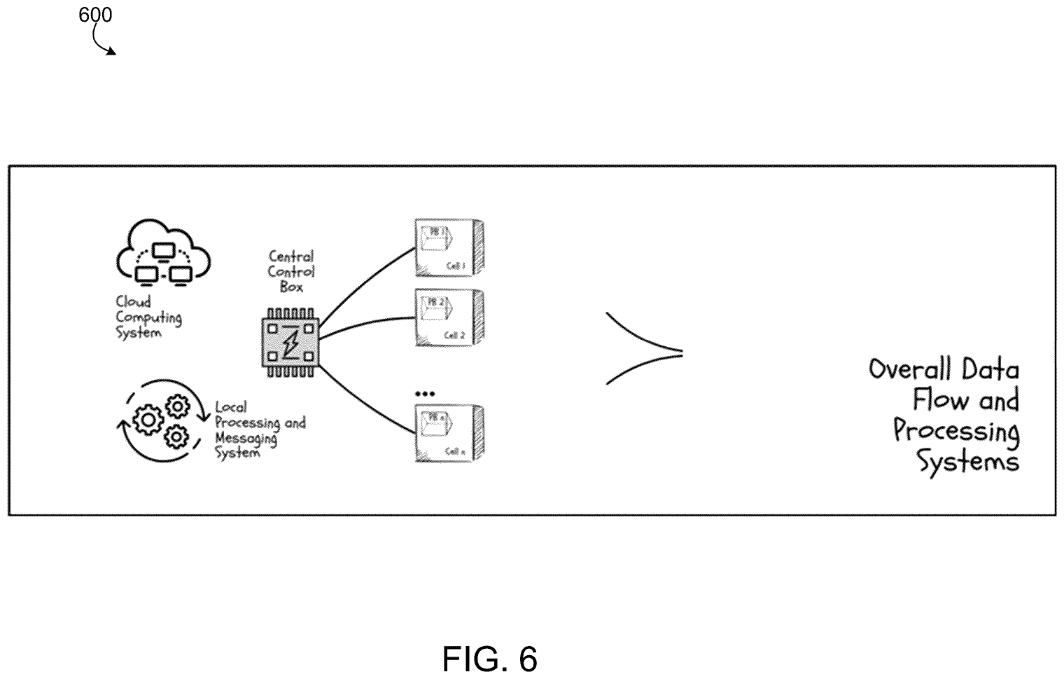

In some implementations, each smart device 108 transmits data in real-time or substantial real time to the monitoring system 102, which acts as the central control unit. The monitoring system 102 can interface with a local processing system, a messaging system, a display, and a secure cloud computing platform, such as that shown in FIG. 6. As a result, this computer structured architecture with the specific smart device 108 enables the detection and differentiation of different health and behavior metric of users monitored by each specific smart device 108. The different health and behavior metrics, as will be further described below, can include, for example, destructive behaviors, self-harm, and medical distress. This detection of the different health and behavior metrics ensures timely interventions while safeguarding privacy and minimizing disruption to existing systems.

FIG. 1 illustrates various operation in stages (A) through (F), which can be performed in a sequence indicated or another sequence. For instance, some of the stages can be performed concurrently, in part of in whole, can be skipped, or can be performed in different orders, e.g., stage (A) can be performed after stage (B).

During stage (A), the different smart devices 108 monitor one or more users in different areas. For example, smart device 108-1 monitors one or more users in area 106-1, smart device 108-2 monitors one or more users in area 106-2, and smart device 106-N monitors one or more users in area 106-N. Each of the one of the users may experiencing different threats, medical risks, health risks, behavioral risks, or other, which are detectable by the combination of the smart device 108-1 and the processes performed by the monitoring system 102. In some cases, the processes performed by the monitoring system 102 may be entirely performed within each of the smart devices 108.

For example, smart device 108-1 can utilize artificial intelligence (AI) powered computer vision algorithms with depth and motion detection to track body positions of each user within the area 106-1. The tracking of body positions can be performed by the smart device 108-1 without interfering with each user's visibility. For example, the sensors within the smart device 108-1 can employ pose estimation and action recognition models, such as HRNet and OpenPose, to enable accurate 3D modeling and real-time motion analysis. The infrared and microwave sensors of the smart device 108-1 can, for example, penetrate clothing to detect concealed objects. The concealed objects that may be detected can include objects such as, drugs, weapons, or other illegal objects.

The sensors within each smart device 1081 can utilize anomaly detection models in order to further bolster security. Specifically, the sensors can recognize unusual behavior patterns that often deviate from typical actions. As will be further described, the monitoring system 102 can detect the unusual behavior patterns and trigger alerts for potential drug concealment, potential weapon detection, or ingestion attempts, to name some examples.

In some examples, each smart device 108 can include ultrasonic and radar sensors. The ultrasonic and radar sensors within each smart device 108 can operate within a defined 4.0 m×4.0 m area, for example. These sensors can apply focused beam radar technology to ensure precision, avoiding cross-cell interference with other sensors, and providing high detection capabilities. The positioning of the ultrasonic and radar sensors supports the use of radar technology, avoids cross-cell interference with other sensors, and providing high detection capabilities, while spatial masking prevents false identifications in adjacent cells. By eliminating cross-cell interference using focused-beam radar and spatial masking techniques, this ensures accurate monitoring in confined spaces. The system's ability to track multiple subjects in real time, with AI-driven alerts, improves security response and outperforms traditional systems that may struggle with complex or crowded environments. To enhance the accuracy within the specified area monitored by the ultrasonic and radar sensors, each smart device 108 can apply techniques such as beamforming and sensor fusion.

In some implementations, each smart device 108 can track one or more individuals simultaneously. Each smart device can track these individuals simultaneously using one or more advanced object detection algorithms, such as YOLOv8. This specific algorithm, for example, allows independent monitoring of multiple individuals even with overlapping movements from these individuals. The control unit of each smart device 108 can fuse data together from the thermal cameras and the microwave sensors to provide reliable detection in shared spaced, such as area 106-1.

In some implementations, each smart device 108 is designed with specific materials. For example, the smart device 108 is designed using Aluminum Composite Panels (ACM) or Polycarbonate for the body. Other materials are also possible. The ACM material offers high durability and resistance to impact, making the ACM material worth using for the smart device 108's construction in order to withstand attempts at destruction, despite the ACM material being more expensive than polycarbonate. Polycarbonate, for example, while slightly less robust than ACM, is still highly resistant to damage, is lightweight, and is cost-effective. As a result, both materials, e.g., the ACM material or polycarbonate, are capable of maintaining the structural integrity of the smart device 108 under potential physical stress, ensuring a long-lasting and secure solution. This type of material is beneficial to ensure the smart device 108's components are protected from any damage by a user or from being dropped.

In some implementations, the front panel of the smart device 108 includes reinforced polycarbonate and fiberglass. The reinforced polycarbonate and fiberglass provide high impact resistance and protection, which is crucial for preventing detainee tampering or destruction. However, the reinforced polycarbonate and the fiberglass materials may interfere with certain sensor functions, especially those relying on infrared or thermal detection, as those material can block these sensor specific materials. To mitigate this, the material design of the smart device 108 can incorporate infrared-transmissive windows or transparent polycarbonate sections where needed. The infrared transmissive windows and the transparent polycarbonate sections ensure that radar, ultrasonic waves, and other waves from the sensors can operate effectively behind the front panel. This ensures the combination of materials provides a secure solution between strength of the smart device 108, low cost of the materials, sensor compatibility to avoid interference, and configuration of the sensors within the smart device 108.

In some implementations, the smart device 108 can include multiple sensors. Each sensor within the smart device 108 works collectively to detect and prevent dangerous behaviors and health emergencies. Through AI-enhanced sensors integrated into the smart device 108, the system can captures data on user movement, vital signs, and environmental factors to ensure safety and timely interventions. Each sensor type is enhanced through one or more AI algorithms to reduce false alarms and increase accuracy in high-risk scenarios, aligning with the essential outcomes of this challenge.

As a result, each sensor within the smart device 108 is designed to detect behaviors and health conditions in real-time using AI-driven analytics. These AI enhancements ensure that each Essential Outcome is addressed with the highest level of precision and reliability. To make such a determination, the smart device 108 includes AI enhanced microwave sensors, AI enhanced thermal cameras and passive infrared (PIR) sensors, AI-enhanced ultrasonic sensors, and AI-enhanced sound and noise sensors.

In some implementations, microwave sensing is an effective technology for motion and respiration monitoring. Each smart device 108 can include one or more microwave sensors. Each microwave sensor operates by emitting microwave signals and analyzing the phase and frequency shifts in the reflected signals received by the microwave sensors. The microwave sensors may be, for example, advanced Frequency-Modulated Continuous Wave (FMCW) radars, which provide millimeter-wave resolution. These radars can detect micro-movements such as chest movements during respiration or subtle limb motions, making them particularly effective for safety applications in confined spaces, such as hospitals, mobile units, and detention cells. The FMCW radars can provide high resolution capabilities with a low cost implementation. As a result, the FMCW radars are capable of detecting even sub-millimeter movement through walls, allowing each smart device 108 to perform non-invasive monitoring. Additionally, the smart device 108 can enhance the signals produced by microwave radars with synthetic aperture radar (SAR) imaging, which provides a three-dimensional representation of objects, enabling precise localization and tracking.

In some implementations, each smart device 108 can include one or more thermal cameras. The thermal cameras can detect infrared radiation emitted by objects and living beings. This allows for the smart device 108 to perform non-contact monitoring of a user's body temperature, heart rate via heat-based detection of blood flow, and overall body movement. The thermal cameras can capture high-resolution heatmaps, even in complete darkness, and distinguish the high-resolution heatmaps between heat signatures of different objects and living beings. In some examples, the thermal cameras include wide-angle lenses that cover larger areas with high precision. In some examples, the thermal cameras within each smart device 108 are equipped with uncooled microbolometers, which are highly sensitive and can detect temperature variations as small as 0.1° C. As a result, the use of uncooled microbolometers with the thermal cameras allow the smart device 108 to identify fevers, hypothermia, or abnormal respiration patterns that could indicate distress. In some examples, the control unit within the smart device 108 can apply advanced thermal imaging with noise reduction algorithms to improve signal-to-noise ratios (SNR) in complex environments, such as hospitals or detention centers, in order to ensure better accuracy in secure facilities.

In some implementations, each smart device 108 can include one or more ultrasonic sensors. The ultrasonic sensors operate by emitting high-frequency sound waves and analyzing the reflected sound to determine distance or detect motion. In a confined space like a detention cell or a hospital, the ultrasonic sensors within each smart device 108 can accurately detect movement towards or away from the corresponding smart device 108. As a result, the ultrasonic sensors within each smart device 108 can detect, for example, a user fall, a user acceleration, a user's movement that indicates self-harm, or a user's movement that tampering with fixtures within the particular monitored area.

In some cases, the ultrasonic sensors within each smart device 108 are miniaturized to fit within the compact form housing of the smart device 108. The ultrasonic sensors may include wide-band ultrasonic sensors, which provide high resolution, which distinguishing between small variations in movement patterns. For example, the ultrasonic sensors within each smart device 108 can detect respiratory rates and heartbeats from a distance by measuring micro-vibrations on the body surface.

In some implementations, each smart device 108 can include one or more acoustic sensors. The one or more acoustic sensors can include sound and noise sensors. The sound and noise sensors can be utilized to detect verbal distress cues, e.g., shouting, gasping, and non-verbal sounds that might indicate dangerous behaviors, e.g., loud bangs or continuous scraping sounds.

The acoustic sensors can include directional microphones that localize the source of sound within the particular location. For example, the acoustic sensors are equipped with Micro-Electro-Mechanical Systems (MEMS) technology, which allow for miniaturization and low-power consumption while maintaining high sensitivity. The acoustic sensors can capture even subtle sounds like gasping, which may indicate a medical emergency. In some examples, the acoustic sensors can perform real-time sound analysis for anomaly detection, particularly in high-noise environments, such as hospitals or detention cells.

In some implementations, the output from each of the sensors are fused together in by a control unit of the smart device 108. The control unit can transmit the fused output from each of the sensors to the monitoring system 102 over network 104. As illustrated in FIG. 1, each smart device 108 transmits sensor data 110-1 through 110-N (collectively “sensor data 110”) to the monitoring system 102 over the network 104. The monitoring system 102 can receive the sensor data 110 and process the sensor data 110.

In some implementations, the smart device 108 combines advanced ultrasonic, infrared, and microwave sensors to monitor health metrics and detect concealed objects in detention cells. The smart device 108's integration into a cohesive system provides for a device that can be mobile and used for real time monitoring. As will be further described below, the fused data output by the smart device 108 is processed by AI-powered behavior analysis using various algorithms for emotion detection, health detection, and behavior detection. This results in a new level of precision and responsiveness for monitoring individuals. Moreover, by incorporating focused-beam radar technology and adapting FMCW radar to avoid cross-cell interference, the smart device 108 ensures accurate monitoring within a defined area. Techniques such as spatial masking and beamforming further enhance the system's ability to focus on specific spaces, preventing false identifications and improving detection in adjacent cells. The integration of multiple sensor types and AI models improves detection capabilities, enabling the smart device 108 to identify concealed objects, monitor multiple individuals, and distinguish between normal and risky behaviors. Real-time alerts and monitoring provide immediate responses to potential issues, while the system's scalability and flexibility allow it to adapt to various contexts, offering a cost-effective and versatile solution.

In some implementations, a Smart Cell provides an approach to detention monitoring. Its integrated system, combining sensors and AI for simultaneous detection of multiple health metrics and behaviors, is not commonly known, or applied in current technologies. The use of advanced AI algorithms for real-time behavior detection, emotion analysis, and anomaly recognition represents a significant breakthrough in detention monitoring solutions.

During stage (B), upon receipt of the sensor data 110 from one or more of the smart devices 108, the monitoring system 102 can provide the sensor data 110 to the calibration and normalization module 112. The calibration and normalization module 112 can perform operations that calibrates the sensor data 110 to be configured as inputs to the trained artificial intelligence models 116. In some cases, the calibration and normalization module 112 can apply one or more noise reduction algorithms to improve the overall SNR of the sensor data 110. In some cases, the calibration and normalization module 112 can normalize the sensor data 110 to filter or smooth random fluctuations in sensor signals or sensor values. Additionally, the calibration and normalization module 112 may normalize the values in the sensor data 110 to the data is comparable and understandable by the trained artificial intelligence models 116.

During stage (C), the calibrated and normalized features are provided to the feature extraction module 114. The feature extraction module 114 can process portions of the calibrated and normalized data to derive features that characterize movement data, thermal data, ultrasonic data, or other data identified in the sensor data 110. For example, the feature extraction module 114 may access portions of the normalized and calibrated sensor data and to identify data values at each of the discrete detection times. The feature extraction module 114 may, in some instances, compute “micro-differences” in the movements shown in the sensor data between each of the discrete detection times. Based on the computed micro-differences, derive values of one or more features that characterize movement of the user, body temperature, heart rate, or other physiological characteristics of the user during the collection period. Other functions of the feature extraction module 114 can be found in U.S. patent application Ser. No. 15/669,316.

During stage (D), the feature extraction module 114 can generate time-varying feature data. The generated feature data includes data that identifies the derived feature values that characterize the information in the sensor data 110. The monitoring system 102 can provide the generated feature data as input to the trained artificial intelligence models 116. The trained artificial intelligence models can determine, from the generated feature data, the health metrics 118 and the behavioral metrics 120 associated with the users monitored by each of the smart devices 108.

In some implementations, the trained artificial intelligence models 116 can include multiple models that process a different portion of the generated feature data. As a result, the monitoring system 102 integrates a series of models to enhance the efficiency of the smart device 108's sensor suite. Thes models include advanced deep learning architectures, reinforcement learning algorithms, and state-of-the-art transformers. For example, the trained artificial intelligence models 116 can include Convolutional Neural Networks (CNNs), Recurrent Neural Networks (RNNs), and reinforcement learning (RL) algorithms.

In some implementations, the CNN and RNN models are employed for real time image and signal analysis. These models allow for the detection of complex behaviors and health indicators. For example, the application of CNNs to thermal and microwave sensor data provides an indication activity recognition and medical distress detection. This can include, for example, whether the user is in motion, performing a particular action, or is in dire need of medical attention.

In some implementations, the trained artificial intelligence models 116 can include transformer models. The transformer models, such as BERT and GPT-based architectures, are used for analyzing audio and visual data from the acoustic sensors within each smart device 108. The transformer models are adept at capturing context and nuances in the acoustic data, which enhances the system's ability to differentiate between normal and abnormal behaviors. For example, the transformer models can recognize emotion and speech recognition tasks in a noisy or clutter environment using their attention mechanisms.

In some implementations, the trained artificial intelligence models 116 can include RL algorithms. The RL algorithms are utilized to optimize sensor configurations and real-time adjustments. These algorithms learn from interactions with the environment to continuously improve the system's response to various scenarios, such as detecting aggression or medical emergencies.

In some implementations, the trained artificial intelligence models 116 can employ Generative Adversarial Networks (GANs). The GANs are used to simulate diverse behavioral scenarios, enabling the system to be trained on a wide range of potential incidents. This approach enhances the model's robustness and generalizability in real-world settings.

In some implementations, the CNN and the RNN models can process data provided by the microwave sensors. CNNs are capable of identifying patterns in Doppler-shifted radar signals to detect specific actions like aggressive motions, falls, or prolonged inactivity, which could indicate a medical emergency.

The RL algorithms are incorporated to optimize settings for each of the sensors in the smart device 108 in real-time. For example, the RL algorithms can produce output that adjust for varying environmental conditions such as changes in humidity or temperature, which could affect the microwave signal. More specifically, the RL algorithms can highlight the use of transformers in radar-based activity recognition, which further improves the detection accuracy by considering the temporal sequence of movements

AI plays a significant role in enhancing the capabilities of thermal cameras. LSTM networks are used to analyze sequences of temperature data over time to detect anomalies such as sudden temperature spikes (fever) or drops (hypothermia). In particular, hybrid AI models combining LSTM and CNN architectures improve the detection of self-harm behavior based on heat patterns (e.g., detecting strangulation or head banging through heat anomalies). Transformer models, known for their attention mechanisms, are increasingly being employed in thermal anomaly detection. These models excel at focusing on critical parts of the heatmap while ignoring irrelevant background noise. Recent studies (Lin et al., 2023) highlight that transformers outperform traditional RNN-based models in terms of accuracy and response time when identifying medical emergencies. GANs have also been employed to enhance the AI's capability to identify simulated dangerous behaviors in a safe, virtual environment before deployment in real-world scenarios.

In some implementations, the trained artificial intelligence models 116 can include Support Vector Machines (SVMs) and Decision Trees. The SVMs and Decision Trees can process data produced by the AI enhances ultrasonic sensors. For instance, an SVM model can differentiate between movements indicating normal behavior and those signaling self-harm or aggression. The monitoring system 102 can train the SVMs and Decision trees using unsupervised learning techniques, such as clustering algorithms, to detect unusual movement patterns that deviate from normal detainee behavior.

In some implementations, the trained artificial intelligence models 116 can include AutoML to automatically select and fine-tune AI models for ultrasonic sensor data. As a result, the AutoML allows the models to adapt to the unique conditions of each area 106. For example, the monitoring system 102 can apply a deep learning framework to the models that uses transfer learning to improve ultrasonic-based activity recognition in environments with domain shift, a common challenge in real-world detention environments where physical settings vary. Moreover, the trained artificial intelligence models 116 can apply reinforcement learning to further optimize sensor positioning and orientation for maximizing detection accuracy in confined spaces.

In some implementations, the trained artificial intelligence models 116 can use Natural Language Processing (NLP) for sound analysis. The NLP can interpret and classify different types of vocalizations from the acoustic sensor data. For example, the NLP models can rely on advanced models such as BERT (Bidirectional Encoder Representations from Transformers). The BERT models can analyze the context and emotion behind spoken words, helping to distinguish between normal speech and distress signals. Moreover, the NLP techniques can identify patterns in non-verbal sounds that may indicate aggressive behavior or self-harm.

In some implementations, the trained artificial intelligence models 116 can utilize transformer models and attention mechanisms to improv the accuracy of sound-based anomaly detection. For example, the transformer models and attention mechanism can process complex sound patterns and enhance emotion recognition in noisy environments. The models can leverage deep neural networks (DNNs), which further refines the detection of vocal distress or aggression, providing actionable insights to intervention teams.

In some implementations, the trained artificial intelligence models 116 can utilize unsupervised learning and temporal pattern recognition to detect escalating threats or health risks. The models 116 detect the escalating threats or health risks by identifying patterns like repetitive motions linked to drug preparation. In some examples, these models 116 can detect stress or anxiety from user movements, providing real-time alerts for enhanced situational awareness.

The trained intelligence models 116 can output the health metrics 118 and the behavioral metrics 120. The health metrics 118 can include vital signs such as heart rate, respiration, and body temperature, to name some examples. The health metrics 118 can include indications of identified abnormal patterns, such as severe fluctuations in heart rate or breathing irregularities, signal medical distress. The behavioral metrics 120 can include, for example, destructive behaviors or self-harm behaviors. Microwave sensors, sound and noise sensors, and thermal cameras incorporated in each smart device 108 detect and analyze unusual movement patterns and temperature changes indicative of destructive actions. The destructive behaviors can include normal behaviors, aggressive behaviors, unusual movement patterns, and temperature changes indicative of destructive behaviors, such as lighting fires or fighting with high precision detected by microwave radars, to name some examples. The self-harm behaviors can include heat patterns associated with self-harm, such as strangulation or head banging. Thermal cameras combined with LSTM networks can identify the heat patterns associated with the self-harm. The LSTM networks can analyze changes in body temperature and movement to detect such behaviors accurately.

In some implementations, the trained artificial intelligence models 116 can apply one or more models for pose estimation and action recognition. For example, the trained artificial intelligence models 116 can include HRNet for pose estimation and OpenPose for action recognition, allowing for an analysis of user behavior in real-time with high accuracy. Transformer-based models for emotion detection add a layer of insight into detainee stress or anxiety, providing situational awareness that current technologies cannot match.

The health metrics 118 and behavioral metrics 120 may include a statistical likelihood indicating how likely a corresponding health or behavioral metric occurs. For example, the health metrics 118 can include an indication of a 90% confidence of a 65 beats per minute (BPM) heart rate. The behavioral metrics 120 can include an indication of 65% confidence that a user detected by the smart device 108-2 is currently performing a self-harm act. The likelihoods can be associated with each of the aforementioned metrics.

During stage (E), the monitoring system 102 can provide the health metrics 118 and the behavioral metrics 120 to a display 122. The display 122 can present the health metrics 118 and the behavioral metrics 120 for each user monitored in each of the areas 106 in real time. If the monitoring system 102 determines there is an update to one of the health metrics 118 or the behavioral metrics 120, then the monitoring system 102 can update the corresponding metric on the display 122 in real time. For example, the display 122 in FIG. 1 illustrates that the monitoring system 102 is 65% confident that user 1 is currently illustrating self-harm, 80% confident that user 2 is under medical distress, and 100% confident that user N is currently asleep.

During stage (F), the monitoring system 102 can determine whether to execute one or more actions to mitigate any issues identified by a smart device and the corresponding monitoring system 102. In particular, the monitoring system 102 can compare the likelihoods for each of the health metrics 118 and the behavioral metrics 120 to user defined thresholds. In some examples, the monitoring system 102 can define the thresholds. In some example, each user in the areas 106 can set user defined threshold for the health metrics 118 and the behavioral metrics 120. Medical personnel can adjust heart rate and breath rate thresholds based on individual detainee needs through the monitoring system 102 software, by accommodating pre-existing medical conditions.

If the monitoring system 102 determines that a likelihood for at least one of the health metrics 118 and the behavioral metrics 120 does not satisfy a threshold value, then the monitoring system 102 can execute one or more actions to mitigate a health or behavioral issue. In some examples, the monitoring system 102 may alert emergency services that a user needs medical services. In some examples, the monitoring system 102 may open a door to an area where a corresponding user was found to need medical attention or protection from self-harm. In some examples, the monitoring system 102 may flicker a light off and on to determine whether the user in Area 106-N is asleep after not having moved for a predetermined period of time. Other examples also possible. In some examples, if the monitoring system 102 determines that each likelihood for the at health metrics 118 and the behavioral metrics 120 does satisfy a threshold value, then the monitoring system 102 does not perform an action to address the health or behavioral issue.

In some implementations, the monitoring system 102 can record and retain the sensor data 110 locally for post-incident analysis. The retained sensor data 110 can be used to train new artificial intelligence models or for retraining models that require updates. The monitoring system 102 can record and retain the health metrics 118 and the behavioral metrics 120 associated with the sensor data 110. In some cases, the monitoring system 102 can store any data received, processed, or produced by the monitoring system 102 in a database, data store, or other device that is locally connected or connected over a network to the monitoring system 102.

In some implementations, the monitoring system 102 can support centralized monitoring for multiple areas. For example, the monitoring system 102 can support centralized monitoring for 2, 4, 6, 8, 10, or a greater number of areas through a single graphical user interface (GUI) of the display 122. Each graphical data fusion on the GUI can display real-time sensor data for each cell. This scalable architecture allows easy integration of additional areas, with individual real-time feeds for efficient monitoring without overwhelming users that monitor the display 122.

In some implementations, the areas 106 can be adapted to a variety of environments. These environments include, for example, transportation vehicles, hospitals, schools, or other areas. Drawing on scalable microwave security system architectures, the system 100 can be reconfigured for smaller or differently shaped spaces. Mobile units employ compact sensors while retaining detection algorithms for posture and anomalous behavior, with device-agnostic AI models allowing flexibility across hardware setups.

The monitoring system 102 can achieve 80-85% detection accuracy using transformer models for anomaly detection, supported by ensemble learning techniques like Random Forest and Gradient Boosting Machines, which enhance prediction reliability and reduce false positives. Calibration data helps refine algorithms, while active learning models ensure continuous improvement in radar activity recognition.

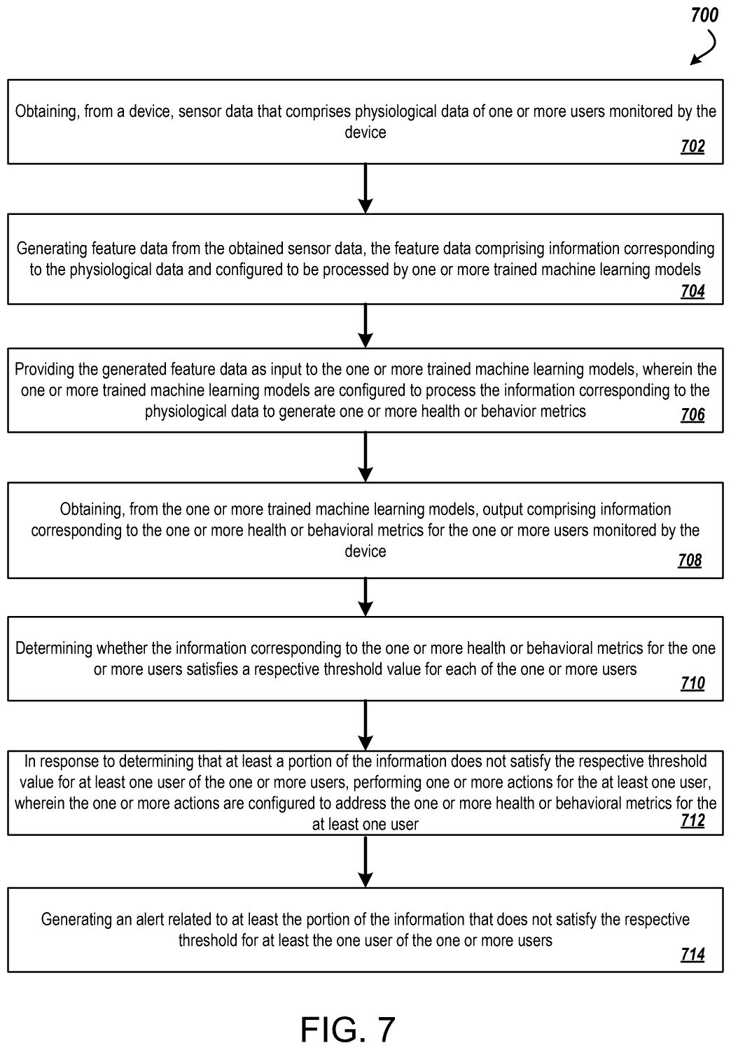

FIG. 7 is a flow diagram that illustrates a process 700 for detecting an individual's physiological condition within an environment using the smart electronic device. For convenience, the process 700 will be described as being performed by a system of one or more computers, located in one or more locations, and programmed appropriately in accordance with this specification. For example, the monitoring system 102 of FIG. 1, appropriately programmed, can perform the process 700.

The system obtains, from a device, sensor data that includes physiological data of one or more users monitored by the device (702). The device includes a multi-sensor device that is configured to (i) detect concealed objects on the one or more users, (ii) track body posture of the one or more users, and (iii) tracking the physiological data of the one or more users. The physiological data of the one or more users includes a breathing rate, a heart rate, a respiration rate, and a body temperature.

The system generates feature data from the obtained sensor data, the feature data including information corresponding to the physiological data and configured to be processed by one or more trained machine learning models (704). The system generates feature data by applying one or more noise reduction algorithms to the obtained sensor data to reduce noise in the obtained sensor data that was created from an area outside where the one or more users are being monitored by the device.

The system provides the generated feature data as input to the one or more trained machine learning models, the one or more trained machine learning models are configured to process the information corresponding to the physiological data to generate one or more health or behavior metrics (706). This includes, for example, the system providing the generated feature data as input to at least one of a convolutional neural network (CNN), a reinforcement learning (RL) algorithm, or a long-short term memory (LSTM) model. The CNN is configured to detect one or more actions indicative of a medical emergency associated with the one or more users using the generated feature data. The RL algorithm is configured to generate feedback to improve conditions of one or more sensors of the device according to detected environmental conditions in the generated feature data. The LSTM model is configured to detect temperature anomalies in the generated feature data.

The system obtains, from the one or more trained machine learning models, output including information corresponding to the one or more health or behavioral metrics for the one or more users monitored by the device (708). Obtaining the output includes the system obtaining data indicative of one or more of destructive behaviors, self-harm behaviors, or medical distress for each user of the one or more users.

The system determines whether the information corresponding to the one or more health or behavioral metrics for the one or more users satisfies a respective threshold value for each of the one or more users (710). For instance, the system obtains a likelihood for each of the one or more health or behavioral metrics for each of the one or more users. The system retrieves one or more thresholds for each of the one or more users. The system compares the likelihood for each of the one or more health or behavioral metrics for each of the one or more users to the respective threshold from the one or more thresholds.

In some cases, in response to comparing, the system can determine that the likelihood of at least one of the one or more health or behavioral metrics for a user does not satisfy the respective threshold for the user. In some cases, in response to comparing, the system can determine that the likelihood for each of the one or more health or behavioral metrics does satisfy the respective threshold from the one or more thresholds.

In response to determining that at least a portion of the information does not satisfy the respective threshold value for at least one user of the one or more users, the system can perform one or more actions for the at least one user, wherein the one or more actions are configured to address the one or more health or behavioral metrics for the at least one user (712). These actions can include, for example, opening a door at a location where the at least one user is being monitored, repeatedly turning a light off and on at the location where the at least one user is being monitored, transmitting a notification to the authorities to alert of an issue associated with the at least one user, or providing a message to the device that is monitoring the at least one user to cause the device to output an audible message through a speaker of the device. Other examples for alerting, notifying, or providing attention to the at least one user are also possible.

The system generates an alert related to at least the portion of the information that does not satisfy the respective threshold for at least the one user of the one or more users (714). The alert includes, for example, data identifying the one or more users, the sensor data of the one or more users, or the obtained information corresponding to the one or more health or behavior metrics for the one or more users monitored by the corresponding device. In some examples, generating the alert includes displaying the alert related to tracking the one or more users monitored by the device.

This specification uses the term “configured” in connection with systems and computer program components. For a system of one or more computers to be configured to perform particular operations or actions means that the system has installed thereon software, firmware, hardware, or a combination thereof that, in operation, cause the system to perform the operations or actions. For one or more computer programs to be configured to perform particular operations or actions means that the one or more programs include instructions that, when executed by data processing apparatus, cause the apparatus to perform the operations or actions.

Implementations of the subject matter and the functional operations described in this specification can be realized in digital electronic circuitry, in tangibly-embodied computer software or firmware, in computer hardware, including the structures disclosed in this specification and their structural equivalents, or in combinations of one or more of them. Implementations of the subject matter described in this specification can be implemented as one or more computer programs (i.e., one or more modules of computer program instructions) encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. The program instructions can be encoded on an artificially-generated propagated signal (e.g., a machine-generated electrical, optical, or electromagnetic signal) that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include special purpose logic circuitry (e.g., an FPGA (field programmable gate array) or an ASIC (application-specific integrated circuit)). The apparatus can optionally include, in addition to hardware, code that creates an execution environment for computer programs (e.g., code) that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data (e.g., one or more scripts stored in a markup language document) in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in some cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry (e.g., a FPGA, an ASIC), or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data (e.g., magnetic, magneto-optical disks, or optical disks). However, a computer need not have such devices. Moreover, a computer can be embedded in another device (e.g., a mobile telephone, a personal digital assistant (PDA), a mobile audio or video player, a game console, a Global Positioning System (GPS) receiver), or a portable storage device (e.g., a universal serial bus (USB) flash drive) to name just a few.

Computer-readable media suitable for storing computer program instructions and data include all forms of non-volatile memory, media, and memory devices, including by way of example semiconductor memory devices (e.g., EPROM, EEPROM, and flash memory devices), magnetic disks (e.g., internal hard disks or removable disks), magneto-optical disks, and CD-ROM and DVD-ROM disks.

To provide for interaction with a user, implementations of the subject matter described in this specification can be provisioned on a computer having a display device (e.g., a CRT (cathode ray tube) or LCD (liquid crystal display) monitor) for displaying information to the user and a keyboard and a pointing device (e.g., a mouse, a trackball), by which the user can provide input to the computer. Other kinds of devices can be used to provide for interaction with a user as well; for example, feedback provided to the user can be any form of sensory feedback (e.g., visual feedback, auditory feedback, tactile feedback); and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device (e.g., a smartphone that is running a messaging application), and receiving responsive messages from the user in return.

Data processing apparatus for implementing machine learning models can also include, for example, special-purpose hardware accelerator units for processing common and compute-intensive parts of machine learning training or production (i.e., inference, workloads).

Machine learning models can be implemented and deployed using a machine learning framework (e.g., a TensorFlow framework, a Microsoft Cognitive Toolkit framework, an Apache Singa framework, an Apache MXNet framework).

Implementations of the subject matter described in this specification can be realized in a computing system that includes a back-end component (e.g., as a data server) a middleware component (e.g., an application server), and/or a front-end component (e.g., a client computer having a graphical user interface, a web browser, or an app through which a user can interact with implementations of the subject matter described in this specification), or any combination of one or more such back-end, middleware, or front-end components. The components of the system can be interconnected by any form or medium of digital data communication (e.g., a communication network). Examples of communication networks include a local area network (LAN) and a wide area network (WAN) (e.g., the Internet).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a user device (e.g., for purposes of displaying data to and receiving user input from a user interacting with the device), which acts as a client. Data generated at the user device (e.g., a result of the user interaction) can be received at the server from the device.

While this specification contains many specific implementation details, these should not be construed as limitations on the scope of any invention or on the scope of what may be claimed, but rather as descriptions of features that may be specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially be claimed as such, one or more features from a claimed combination can in some cases be excised from the combination, and the claimed combination may be directed to a sub-combination or variation of a sub-combination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the implementations described above should not be understood as requiring such separation in all implementations, and it should be understood that the described program components and systems can generally be integrated together in a single software product or packaged into multiple software products.

Particular implementations of the subject matter have been described. Other implementations are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method comprising:
obtaining, from a device, sensor data that comprises physiological data of one or more users monitored by the device;
generating feature data from the obtained sensor data, the feature data comprising information corresponding to the physiological data and configured to be processed by one or more trained machine learning models;
providing the generated feature data as input to the one or more trained machine learning models, wherein the one or more trained machine learning models are configured to process the information corresponding to the physiological data to generate one or more health or behavior metrics;
obtaining, from the one or more trained machine learning models, output comprising information corresponding to the one or more health or behavioral metrics for the one or more users monitored by the device;
determining whether the information corresponding to the one or more health or behavioral metrics for the one or more users satisfies a respective threshold value for each of the one or more users;
in response to determining that at least a portion of the information does not satisfy the respective threshold value for at least one user of the one or more users, performing one or more actions for the at least one user, wherein the one or more actions are configured to address the one or more health or behavioral metrics for the at least one user; and
generating an alert related to at least the portion of the information that does not satisfy the respective threshold for at least the one user of the one or more users.

2. The method of claim 1, wherein obtaining sensor data that comprises physiological data of one or more users monitored by the device comprises obtaining, from the device, the sensor data that comprises the physiological data of the one or more users monitored by the device, wherein the device comprises a multi-sensor device that is configured to (i) detect concealed objects on the one or more users, (ii) track body posture of the one or more users, and (iii) tracking the physiological data of the one or more users.

3. The method of claim 2, wherein the physiological data of the one or more users comprises a breathing rate, a heart rate, a respiration rate, and a body temperature.

4. The method of claim 1, wherein generating feature data from the obtained sensor data comprises applying one or more noise reduction algorithms to the obtained sensor data to reduce noise in the obtained sensor data that was created from an area outside where the one or more users are being monitored by the device.

5. The method of claim 1, wherein providing the generated feature data as input to the one or more trained machine learning models comprises:
providing the generated feature data as input to at least one of a convolutional neural network (CNN), a reinforcement learning (RL) algorithm, or a long-short term memory (LSTM) model,
wherein the CNN is configured to detect one or more actions indicative of a medical emergency associated with the one or more users using the generated feature data,
wherein the RL algorithm is configured to generate feedback to improve conditions of one or more sensors of the device according to detected environmental conditions in the generated feature data, and
wherein the LSTM model is configured to detect temperature anomalies in the generated feature data.

6. The method of claim 1, wherein obtaining, from the one or more trained machine learning models, output comprising information corresponding to the one or more health or behavioral metrics for the one or more users monitored by the device comprises obtaining data indicative of one or more of destructive behaviors, self-harm behaviors, or medical distress for each user of the one or more users.

7. The method of claim 6, wherein determining whether the information corresponding to the one or more health or behavioral metrics for the one or more users satisfies a respective threshold for each of the one or more users comprises:
obtaining a likelihood for each of the one or more health or behavioral metrics for each of the one or more users;
retrieving one or more thresholds for each of the one or more users; and
comparing the likelihood for each of the one or more health or behavioral metrics for each of the one or more users to the respective threshold from the one or more thresholds.

8. The method of claim 7, further comprising:
determining that the likelihood of at least one of the one or more health or behavioral metrics for a user does not satisfy the respective threshold for the user; or
determining that the likelihood for each of the one or more health or behavioral metrics does satisfy the respective threshold from the one or more thresholds.

9. The method of claim 1, wherein performing one or more actions for the at least one user comprises:
opening a door at a location where the at least one user is being monitored;
repeatedly turning a light off and on at the location where the at least one user is being monitored;
transmitting a notification to the authorities to alert of an issue associated with the at least one user; or
providing a message to the device that is monitoring the at least one user to cause the device to output an audible message through a speaker of the device.

10. The method of claim 1, wherein the alert comprises data identifying the one or more users, the sensor data of the one or more users, or the obtained information corresponding to the one or more health or behavior metrics for the one or more users monitored by the corresponding device.

11. The method of claim 1, wherein generating the alert comprises displaying the alert related to tracking the one or more users monitored by the device.

12. A device comprising:
one or more thermal cameras configured to generate thermal data of one or more users in a location monitored by the device;
one or more ultrasonic sensors configured to analyze sound reflections to detect movement of the one or more users in the location;
one or more infrared sensors configured to monitor physiological data of the one or more users;
one or more microwave sensors configured to generate motion data and the physiological data of the one or more users; and
a control unit configured to:
generate combined data comprising the thermal data, the detected movement, the physiological data, and the motion data; and
generate one or more messages characterizing the combined data.

13. The device of claim 12, further comprising one or more acoustic sensors configured to record noise and sounds at the location of the one or more users.

14. The device of claim 12, further comprising a housing configured to house the one or more thermal cameras, the one or more ultrasonic sensors, the one or more infrared sensors, the one or more microwave sensors, and the control unit.

15. The device of claim 14, wherein a front panel of the housing comprises at least one of:
a transparent polycarbonate material configured to allow infrared transmissions,
reinforced fiberglass, or
reinforced polycarbonate.

16. The device of claim 15, wherein the transparent polycarbonate material is a one-way transparent material.

17. The device of claim 15, wherein the housing comprises at least one of Aluminum Composite Panels (ACM) or Polycarbonate.

18. The device of claim 15, wherein the housing comprises a right triangular pentahedron prism.

19. The device of claim 18, wherein the right triangular pentahedron prism is configured to mount to a wall-ceiling junction.

20. A non-transitory computer-readable medium storing software comprising instructions executable by one or more computers which, upon such execution, cause the one or more computers to perform operations comprising:
obtaining, from a device, sensor data that comprises physiological data of one or more users monitored by the device;
generating feature data from the obtained sensor data, the feature data comprising information corresponding to the physiological data and configured to be processed by one or more trained machine learning models;
providing the generated feature data as input to the one or more trained machine learning models, wherein the one or more trained machine learning models are configured to process the information corresponding to the physiological data to generate one or more health or behavior metrics;
obtaining, from the one or more trained machine learning models, output comprising information corresponding to the one or more health or behavioral metrics for the one or more users monitored by the device;
determining whether the information corresponding to the one or more health or behavioral metrics for the one or more users satisfies a respective threshold value for each of the one or more users;
in response to determining that at least a portion of the information does not satisfy the respective threshold value for at least one user of the one or more users, performing one or more actions for the at least one user, wherein the one or more actions are configured to address the one or more health or behavioral metrics for the at least one user; and
generating an alert related to at least the portion of the information that does not satisfy the respective threshold for at least the one user of the one or more users.
